(19) United States
(12) Patent Application Publication
(10) Pub. No.: US 2013/0295692 A1
(43) Pub. Date: Nov. 7, 2013

(54) MEANS AND METHODS FOR PREDICTING DIABETES

(71) Applicants: Jan C. Wiemer, Berlin (DE); Dietrich Rein, Berlin (DE); Inken Padberg, Berlin (DE); Oliver Schmitz, Dallgow-Doberitz (DE); Volker Liebenberg, Berlin (DE); Victoria Nikiforova, Werder (DE)

(72) Inventors: Jan C. Wiemer, Berlin (DE); Dietrich Rein, Berlin (DE); Inken Padberg, Berlin (DE); Oliver Schmitz, Dallgow-Doberitz (DE); Volker Liebenberg, Berlin (DE); Victoria Nikiforova, Werder (DE)

(73) Assignee: METANOMICS HEALTH GMBH, BERLIN (DE)

(21) Appl. No.: 13/996,742

(22) PCT Filed: Dec. 23, 2011
(86) PCT No.: PCT/IB11/55935

§ 371 Date: Jul. 9, 2013

Related U.S. Application Data
(60) Provisional application No. 61/426,549, filed on Dec. 23, 2010.

(30) Foreign Application Priority Data
Dec. 23, 2010 (EP) 10196869.1

(51) Int. Cl. G01N 33/53

(52) U.S. Cl. CPC G01N 33/53
USPC 436/501; 422/69

(57) ABSTRACT

A method for diagnosing diabetes or a predisposition for diabetes is provided, which comprises determining the amount of glyoxylate in a test sample of a subject suspected to suffer from diabetes or to have a predisposition for diabetes and comparing said amount to a references, whereby diabetes or a predisposition for diabetes is to be diagnosed. Further, the use of glyoxylate or a detection agent for glyoxylate for diagnosing diabetes or a predisposition for diabetes is provided. Moreover, a device and a kit for diagnosing diabetes or a predisposition for diabetes are also provided.

The present invention relates to the field of diabetes diagnostics. In particular, it relates to a method for diagnosing diabetes or a predisposition for diabetes comprising determining the amount of glyoxylate in a test sample of a subject suspected to suffer from diabetes or to have a predisposition for diabetes and comparing said amount to a reference, whereby diabetes or a predisposition for diabetes is to be diagnosed. Further, the present invention relates to the use of glyoxylate or a detection agent for glyoxylate for diagnosing diabetes or a predisposition therefor in a sample of a subject Moreover, the present invention encompasses a device and a kit for diagnosing diabetes or a predisposition therefor.

The prevalence of diabetes mellitus has reached about 6% in the industrialised world and will increase up to 366 million affected people in 2030 worldwide. The most frequent reason (type), (about 90%) for diabetes in the world is accounted for type 2 diabetes, which has a multifactorial pathogenesis. The pathological sequence for type 2 diabetes entails many elements. It is believed to be mandatory to have a genetic predisposition that is currently poorly understood. Whether the diabetes phenotype then occurs is influenced by many environmental factors that share an ability to stress the glucose homeostasis system, either by causing or worsening insulin resistance or impairing insulin secretion. Of course many hormones are taking part in the regulation of glucose metabolism, but the key hormone is insulin. Normoglycaemia is maintained by the balanced interplay between insulin action and insulin secretion. Insulin is produced by the pancreatic β-cell which is able to regulate very fast to different glucose demands. The main reason for type 2 diabetes is an increasing insulin resistance. Therefore, insulin action normally decrease but initially the system is able to compensate this by an increasing β-cell function. At this time perhaps only an impaired fasting glucose or an impaired glucose tolerance in the OGTT could be measured. But over time the β-cell will be overstressed by increasing insulin resistance and glucose toxicity and a type 2 diabetes could be diagnosed.

Apart from direct medical problems by high or low blood sugar the main medical and socioeconomic burden of the disease is caused by the associated complications. The devastating complications of diabetes mellitus are mostly macrovascular and microvascular diseases like chronic renal failure, retinopathy, periphery and autonomic neuropathy or myocardial infarction. Therefore, cardiovascular morbidity in patients with type 2 diabetes is two to four times greater than that of non-diabetic people (Stumvoll et al., Type 2 diabetes: principles of pathogenesis and therapy, Lancet 2005).

In light of this mechanism, therapy of diabetes is currently based on monitoring the blood sugar levels and reducing an elevated level of blood sugar into a normal level by administration of exogenous insulin. To this end, exogenous insulin is injected into the blood. Alternatively, glucose levels in the blood may be regulated by nutritional diets and the exclusion of life-style risk factors, such as smoking, lack of exercise, high cholesterol levels, and an unstable body weight.

The Expert Committee of the ADA (American Diabetes Association) recognized an intermediate group of subjects whose glucose levels, although not meeting criteria for diabetes, are nevertheless too high to be considered normal. This group is defined as having fasting plasma glucose (FPG) levels>100 mg/dl (5.6 mmol/l) but <126 mg/dl (7.0 mmol/l) or 2-h values in the oral glucose tolerance test (OGTT) of >140 mg/dl (7.8 mmol/l) but <200 mg/dl (11.1 mmol/l). Thus, the categories of FPG values are as follows:
FPG<100 mg/dl (5.6 mmol/l)=normal fasting glucose;
FPG 100-125 mg/dl (5.6-6.9 mmol/l)=IFG (impaired fasting glucose);
FPG>126 mg/dl (7.0 mmol/l)=provisional diagnosis of diabetes (the diagnosis must be confirmed, as described below).

The corresponding categories when the OGTT is used are the following:
2-h postload glucose<140 mg/dl (7.8 mmol/l)=normal glucose tolerance
2-h postload glucose 140-199 mg/dl (7.8-11.1 mmol/l)=IGT (impaired glucose tolerance)
2-h postload glucose>200 mg/dl (11.1 mmol/l)=provisional diagnosis of diabetes (the diagnosis must be confirmed, as described below).

Diagnosis of Diabetes mellitus type 2: Symptoms of diabetes plus casual plasma glucose concentration>200 mg/dl (11.1 mmol/l). Casual is defined as any time of day without regard to time since last meal. The classic symptoms of diabetes include polyuria, polydipsia, and unexplained weight loss. Alternatively: 2. FPG>126 mg/dl (7.0 mmol/l). Fasting is defined as no caloric intake for at least 8 h. Alternatively: 3. 2-h postload glucose>200 mg/dl (11.1 mmol/l) during an OGTT. The test should be performed as described by WHO, using a glucose load containing the equivalent of 75 g anhydrous glucose dissolved in water.

In the absence of unequivocal hyperglycemia, these criteria should be confirmed by repeat testing on a different day. The third measure (OGTT) is not recommended for routine clinical use. (American Diabetes Association, Diagnosis and Classification of Diabetes Mellitus, Diabetes Care 2006) However, an increase in the blood sugar levels or a decrease in the available insulin are developments which are rather downstream events in the development and progression of diabetes.

Diagnostic biomarkers for diabetes have been recently reported (see WO2007/110357; WO2007/110358; WO2009/14639; and WO2010/114897). In urine, an increase of the excretion of glyoxylate has been reported (Yamaguchi 1968, Journal of Osaka City Medical Center 17(9-10): 383-389). However, there is still a need for reliable biomarkers which can be used to identify individuals at risk before the early onset of the disease or at least in an early stage.

Accordingly, the technical problem underlying the present invention must be seen as the provision of means and methods for efficiently and reliably diagnosing diabetes or a predisposition for diabetes. The technical problem is solved by the embodiments characterized in the claims and described herein below.

The present invention relates to a method for diagnosing diabetes or a predisposition therefor comprising:
(a) determining the amount of glyoxylate in a test sample of a subject suspected to suffer from diabetes or to have a predisposition therefor; and
(b) comparing the amount determined in step (a) to a reference, whereby diabetes or a predisposition therefor is to be diagnosed.

The method referred to in accordance with the present invention may essentially consist of the aforementioned steps or may include further steps. Further steps may relate to sample pretreatment or evaluation of the diagnostic results obtained by the method. Preferred further evaluation steps are described elsewhere herein. The method may partially or entirely by assisted by automation. For example, step a) can be automated by robotic and automated reader devices. Step b) can be automated by suitable data processing devices, such as a computer, comprising a program code which when being executed carries out the comparison automatically. A reference in such a case will be provided from as a stored reference, e.g., from a database. It is to be understood that the method is, preferably, a method carried out ex vivo on a sample of a subject, i.e. not practised on the human or animal body.

The term “diagnosing” as used herein refers to assessing the probability according to which a subject is suffering from diabetes or has a predisposition for diabetes. Diagnosis of a predisposition may sometimes be referred to as prognosis or prediction of the likelihood that a subject will develop the disease. As will be understood by those skilled in the art, such an assessment, although preferred to be, may usually not be correct for 100% of the subjects to be diagnosed. The term, however, requires that a statistically significant portion of subjects can be identified as suffering from diabetes or having a predisposition for diabetes. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, 0.05.

Diagnosing according to the present invention also includes monitoring, confirmation, and classification of diabetes or its symptoms as well as a predisposition therefor. Monitoring refers to keeping track of an already diagnosed diabetes or predisposition. Monitoring encompasses, e.g., determining the progression of the disease or predisposition, determining the influence of a particular treatment on the progression of the disease or the influence of prophylactic measures such as a prophylactic treatment or diet on disease development in a subject having a predisposition. Confirmation relates to the strengthening or substantiating a diagnosis of diabetes or a predisposition for diabetes already performed using other indicators or markers. Classification relates to allocating the disease into different disease classes, e.g., corresponding to the strength of the symptoms accompanying the disease, or differentiating between different stages or medical conditions. A predisposition for the disease can be classified based on the degree of the risk, i.e. the probability according to which a subject will develop the disease later. Moreover, the method can also encompass the identification of subjects which develop certain co-morbidities, such as increased blood pressure, or which are at risk for such co-morbidities.

Preferably, subjects can be classified and allocated into different risk groups based on glyoxylate as a biomarker by the method according to the present invention (see tables, below). In particular, glyoxylate is indicative for subjects having an increased risk for diabetes and falling into the risk group of impaired fasting glucose (IFG), impaired glucose tolerance (IGT) or both (IFG&IGT) as indicated in the Tables, below. Thus, preferably, the method of the present invention is a method for diagnosing whether a subject has a predisposition for diabetes or suffers from IFG, IGT or IFG&IGT based on the measurement of glyoxylate.

The term “diabetes” or “diabetes mellitus” as used herein refers to disease conditions in which the glucose metabolism is impaired. Said impairment results in hyperglycaemia. According to the World Health Organisation (WHO), diabetes can be subdivided into four classes. Type 1 diabetes is caused by a lack of insulin. Insulin is produced by the so called pancreatic islet cells. Said cells may be destroyed by an autoimmune reaction in Type 1 diabetes (Type 1a). Moreover, Type 1 diabetes also encompasses an idiopathic variant (Type 1b). Type 2 diabetes is caused by an insulin resistance. Type 3 diabetes, according to the current classification, comprises all other specific types of diabetes mellitus. For example, the beta cells may have genetic defects affecting insulin production, insulin resistance may be caused genetically or the pancreas as such may be destroyed or impaired. Moreover, hormone deregulation or drugs may also cause Type 3 diabetes. Type 4 diabetes may occur during pregnancy. Preferably, diabetes as used herein refers to diabetes Type 2. According to the German Society for Diabetes, diabetes is diagnosed either by a plasma glucose level being higher than 110 mg/dl in the fasting state or being higher than 220 mg/dl postprandial. Further preferred diagnostic techniques are disclosed elsewhere in this specification. Further symptoms of diabetes are well known in the art and are described in the standard text books of medicine, such as Stedman or Pschyrembl.

The term “predisposition” as used herein means that a subject has not yet developed the disease or any of the aforementioned disease symptoms or other diagnostic criteria but, nevertheless, will develop the disease within a defined time window in the future (predictive window) with a certain likelihood. The predictive window is an interval in which the subject shall develop the disease or condition according to the predicted probability. The predictive window may be the entire remaining lifespan of the subject upon analysis by the method of the present invention. Preferably, however, the predictive window is an interval of one month, six months or one, two, three, four, five or ten years after the sample to be analyzed by the method of the present invention has been obtained. The likelihood for developing the diseases referred to herein shall be significantly larger for a subject having a predisposition than the likelihood of statistical appearance of diabetes mellitus within a given cohort of subjects. Preferably, for an individual subject the likelihood associated with a predisposition for developing diabetes is at least 30%, at least 40%, at least 50%, at least 60%, at least 70%, at least 80%, at least 90% or 100% or at least 1.5-times, 2-times, 3-times, 4-times, 5-times or 10-times increased compared with the average likelihood for a subject of a given cohort for developing diabetes. A cohort of subjects as referred to herein means a plurality of individual subjects which are of the same species and, preferably, are of the same or genetic background or ethnical group and, more preferably, also of the same age and gender.

Preferably, said predisposition for diabetes as referred to herein is accompanied by an elevated long-term blood glucose, impaired glucose tolerance (IGT), impaired fasting glucose (IFG) and/or IGT in combination with IFG.

The term “glyoxylate” as referred to herein refers to glyoxylate as naturally occurring or artificially generated derivatives thereof. Naturally occurring derivatives are derivatives which are obtained from glyoxylate by metabolic conversions. Artificially generated derivatives are generated from glyoxylate during the analysis carried out by the method according to the invention, e.g., derivatives which are required for GCMS analysis and the like. It will be understood that the derivatives referred to hereinabove shall reflect the amount of the metabolite found in a subject, i.e. the amount of a derivative determined from a sample of the subject shall correlate with the amount of the metabolite found in the subject at the time when the sample has been taken. The following designations are used synonymously for glyoxylate: glyoxylic acid, formylformate, formylformic acid, glyoxalate, oxalaldehydate, oxalaldehydic acid, oxoacetate, oxoacetatic acid, oxoethanoate, oxoethanoic acid, alpha-ketoacetate or alpha-ketoacetic acid.

In the method of the present invention, at least one further biomarker for diabetes or a predisposition therefor can be determined in addition to glyoxylat. Preferably, said additional at least one biomarker is selected from the group consisting of: cryptoxanthin, 2-hydroxy-palmitic acid, triacylglyceride (C16:0, C18:1, C18:2), gondoic acid, tricosanoic acid, 5-Oxoproline, glucose, haemoglobin HbA1C, 1,5-anhydrosorbitol, 2-hydroxybutyrate, and mannose. Other preferred metabolites to be determined together, i.e. either simultaneously or consecutively, with glyoxylate are metabolite biomarkers indicative for diabetes or a predisposition thereof which are disclosed by WO2007/110357 and WO2007/110358, respectively.

The term “test sample” as used herein refers to samples to be used for the diagnosis of diabetes or a predisposition for diabetes by the method of the present invention. Said test sample is a biological sample. Samples from biological sources (i.e. biological samples) usually comprise a plurality of metabolites. Preferred biological samples to be used in the method of the present invention are samples from body fluids, preferably, blood, plasma, serum, lymph, sudor, saliva, tears, sperm, vaginal fluid, faeces, urine or cerebrospinal fluid, or samples derived, e.g., by biopsy, from cells, tissues or organs. This also encompasses samples comprising subcellular compartments or organelles, such as the mitochondria, Golgi network or peroxisomes. Moreover, biological samples also encompass gaseous samples, such as volatiles of an organism. Biological samples are derived from a subject as specified elsewhere herein. Techniques for obtaining the aforementioned different types of biological samples are well known in the art. For example, blood samples may be obtained by blood taking while tissue or organ samples are to be obtained, e.g., by biopsy. Most preferably, the test sample referred to herein is a blood, plasma or serum sample.

The aforementioned samples are, preferably, pre-treated before they are used for the method of the present invention. As described in more detail below, said pre-treatment may include treatments required to release or separate the compounds or to remove excessive material or waste. Suitable techniques comprise centrifugation, extraction, fractioning, purification and/or enrichment of compounds. Moreover, other pre-treatments are carried out in order to provide the compounds in a form or concentration suitable for compound analysis. For example, if gas-chromatography coupled mass spectrometry is used in the method of the present invention, it will be required to derivatize the compounds prior to the said gas chromatography. Suitable and necessary pre-treatments depend on the means used for carrying out the method of the invention and are well known to the person skilled in the art. Pre-treated samples as described before are also comprised by the term “sample” as used in accordance with the present invention.

The term “subject” as used herein relates to animals, preferably to mammals such as mice, rats, sheep, dogs, cats, horses, monkeys, or cows and, also preferably, to humans. Other animals which may be diagnosed applying the method of the present invention are birds or reptiles. A subject suspected to suffer from diabetes refers to a subject which shows, preferably, symptoms or clinical signs or parameters indicative for diabetes. A subject which has a predisposition for diabetes, preferably, shows no apparent symptoms or clinical signs or parameters indicative for diabetes, i.e. an apparently healthy subject with respect to diabetes. Apparently healthy subjects may be investigated by the method of the present invention also as a measure of preventive care or for population screening purposes. The subject is, preferably, a non-fasting subject at the time when the test sample is to be obtained.

The term “determining” as used herein refers to determining at least one characteristic feature of glyoxylate comprised by the sample referred to herein. Characteristic features in accordance with the present invention are features which characterize the physical and/or chemical properties including biochemical properties of glyoxylate. Such properties include, e.g., molecular weight, viscosity, density, electrical charge, spin, optical activity, elementary composition, chemical structure, capability to react with other compounds, capability to elicit a response in a biological read out system and the like. Values for said properties may serve as characteristic features and can be determined by techniques well known in the art. Moreover, the characteristic feature may be any feature which is derived from the values of the physical and/or chemical properties of glyoxylate by standard operations, e.g., mathematical calculations such as multiplication, division or logarithmic calculus. Most preferably, the at least one characteristic feature allows the determination and/or chemical identification of glyoxylate.

Glyoxylate comprised by a test sample may be determined in accordance with the present invention quantitatively or qualitatively. For qualitative determination, the presence or absence of glyoxylate will be determined by a suitable technique. Moreover, qualitative determination may, preferably, include determination of the chemical structure or composition. For quantitative determination, either the precise amount of glyoxylate present in the sample will be determined or the relative amount thereof will be determined, preferably, based on the value determined for the characteristic feature(s) referred to herein above. The relative amount may be determined in a case were the precise amount glyoxylate can or shall not be determined. In said case, it can be determined whether the amount in which glyoxylate is present is enlarged or diminished with respect to a second sample comprising glyoxylate in a second amount. Quantitatively analysing glyoxylate, thus, also includes what is sometimes referred to as semi-quantitative analysis.

Moreover, determining as used in the method according to the present invention, preferably, includes using a compound separation step prior to the analysis step referred to before. Preferably, said compound separation step yields a time resolved separation of the metabolites comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or GC are chromatographic techniques to be envisaged by the method of the present invention. Suitable devices for such determination of metabolites, such as glyoxylate, are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Most preferably, LC-MS and/or GC-MS are used as described in detail below. Said techniques are disclosed in, e.g., Nissen, Journal of Chromatography A, 703, 1995: 37-57, U.S. Pat. No. 4,540,884 or U.S. Pat. No. 5,397,894, the disclosure content of which is hereby incorporated by reference. As an alternative or in addition to mass spectrometry techniques, the following techniques may be used for compound determination: nuclear magnetic resonance (NMR), magnetic resonance imaging (MRI), Fourier transform infrared analysis (FT-IR), ultra violet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado. The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

As described above, in a preferred embodiment of the method of the present invention, said determining of glyoxylate comprises mass spectrometry (MS).

Mass spectrometry as used herein encompasses all techniques which allow for the determination of the molecular weight (i.e. the mass) or a mass variable corresponding to a compound, i.e. a metabolite, to be determined in accordance with the present invention. Preferably, mass spectrometry as used herein relates to GC-MS, LC-MS, direct infusion mass spectrometry, FT-ICR-MS, CE-MS, HPLC-MS, quadrupole mass spectrometry, any sequentially coupled mass spectrometry such as MS-MS or MS-MS-MS, ICP-MS, Py-MS, TOF or any combined approaches using the aforementioned techniques. How to apply these techniques is well known to the person skilled in the art. Moreover, suitable devices are commercially available. More preferably, mass spectrometry as used herein relates to LC-MS and/or GC-MS, i.e. to mass spectrometry being operatively linked to a prior chromatographic separation step. More preferably, mass spectrometry as used herein encompasses quadrupole MS. Most preferably, said quadrupole MS is carried out as follows: a) selection of a mass/charge quotient (m/z) of an ion created by ionisation in a first analytical quadrupole of the mass spectrometer, b) fragmentation of the ion selected in step a) by applying an acceleration voltage in an additional subsequent quadrupole which is filled with a collision gas and acts as a collision chamber, selection of a mass/charge quotient of an ion created by the fragmentation process in step b) in an additional subsequent quadrupole, whereby steps a) to c) of the method are carried out at least once and analysis of the mass/charge quotient of all the ions present in the mixture of substances as a result of the ionisation process, whereby the quadrupole is filled with collision gas but no acceleration voltage is applied during the analysis. Details on said most preferred mass spectrometry to be used in accordance with the present invention can be found in WO 03/073464.

More preferably, said mass spectrometry is liquid chromatography (LC) MS and/or gas chromatography (GC) MS.

Liquid chromatography as used herein refers to all techniques which allow for separation of compounds (i.e. metabolites including glyoxylate) in liquid or supercritical phase. Liquid chromatography is characterized in that compounds in a mobile phase are passed through the stationary phase. When compounds pass through the stationary phase at different rates they become separated in time since each individual compound has its specific retention time (i.e. the time which is required by the compound to pass through the system). Liquid chromatography as used herein also includes HPLC. Devices for liquid chromatography are commercially available, e.g. from Agilent Technologies, USA. Gas chromatography as applied in accordance with the present invention, in principle, operates comparable to liquid chromatography. However, rather than having the compounds in a liquid mobile phase which is passed through the stationary phase, the compounds will be present in a gaseous volume. The compounds pass the column which may contain solid support materials as stationary phase or the walls of which may serve as or are coated with the stationary phase. Again, each compound has a specific time which is required for passing through the column. Moreover, in the case of gas chromatography it is preferably envisaged that the compounds are derivatised prior to gas chromatography. Suitable techniques for derivatisation are well known in the art. Preferably, derivatisation in accordance with the present invention relates to methoxymation and trimethylsilylation of, preferably, polar compounds and transmethylation, methoxymation and trimethylsilylation of, preferably, non-polar (i.e. lipophilic) compounds.

Moreover, glyoxylate can also be determined by a specific chemical or biological assay. Said assay shall comprise means which allow for specifically detecting glyoxylate in the sample. Preferably, said means are capable of specifically recognizing the chemical structure of glyoxylate or are capable of specifically identifying the glyoxylate based on its capability to react with other compounds or its capability to elicit a response in a biological read out system (e.g., induction of a reporter gene). Means which are capable of specifically recognizing the chemical structure of glyoxylate are detection agents for glyoxylate, preferably, antibodies, proteins or aptamers which specifically bind to gyloxylate. Specific antibodies, for instance, may be obtained using glyoxylate as antigen or from phage antibody libraries by methods well known in the art. Antibodies as referred to herein include both polyclonal and monoclonal antibodies, as well as fragments thereof, such as Fv, Fab and F(ab)₂ fragments that are capable of binding the antigen or hapten. Moreover, encompassed are single chain antibodies and all types of chimeric antibodies. Suitable proteins which are capable of specifically recognizing the glyoxylate are, preferably, enzymes which are involved in the metabolic conversion of the said metabolite. Said enzymes may either use glyoxylate as a substrate or may convert a substrate into the metabolite. Aptameres which specifically bind to glyoxylate can be generated by methods well known in the art (Ellington 1990, Nature 346:818-822; Vater 2003, Curr Opin Drug Discov Devel 6(2): 253-261). Suitable antibody and/or enzyme based assays may be RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA) or solid phase immune tests. Moreover, glyoxylate may also be identified based on its capability to react with other compounds, i.e. by a specific chemical reaction. Further detection methods such as capillary electrophoresis (Hubert 2001, Clinical Chemistry 47: 1319-1321) and colorimetric methods (Kyaw 1978, Clin Chim Acta 86(2):153-7) can be used. Further, glyoxylate may be determined in a sample due to its capability to elicit a response in a biological read out system. The biological response shall be detected as read out indicating the presence and/or the amount of glyoxylate comprised by the sample. The biological response may be, e.g., the induction of gene expression or a phenotypic response of a cell or an organism.

Further, it is to be understood that depending of the technique used for determining the glyoxylate, the analyte which will be detected could be a derivative of the physiologically occurring glyoxylate, i.e. the metabolite present within a subject. Such analytes may be generated as a result of sample preparation or detection means. The compounds referred to herein are deemed to be analytes. However, as set forth above, these analytes will represent glyoxylate in a qualitative and quantitative manner.

The term “reference” refers to an amount of glyoxylate, which can be correlated to diabetes or a predispostion for diabetes. Such a reference is, preferably, obtained from a sample from a subject known to suffer from diabetes or to have a predisposition for diabetes. The reference may be obtained by applying the method of the present invention. Alternatively, but nevertheless also preferred, the reference may be obtained from a sample of a subject known not to suffer from diabetes or not to have a predisposition for diabetes. Moreover, the reference, also preferably, could be a calculated reference, most preferably the average or median, for the relative or absolute amount of glyoxylate of a population of individuals comprising the subject to be investigated, such as a representative cohort of Caucasians. The absolute or relative amounts of glyoxylate of said individuals of the population can be determined as specified elsewhere herein. How to calculate a suitable reference, preferably, the average or median, is well known in the art. Other techniques for calculating a suitable reference include optimization using receiver operating characteristics (ROC) curve calculations which are also well known in the art and which can be performed for an assay system having a given specificity and sensitivity based on a given cohort of subjects without further ado. The population of subjects referred to before shall comprise a plurality of subjects, preferably, at least 5, 10, 50, 100, 1,000 or 10,000 subjects. It is to be understood that the subject to be diagnosed by the method of the present invention and the subjects of the said plurality of subjects are of the same species.

More preferably, the reference will be stored in a suitable data storage medium such as a database and are, thus, also available for future diagnoses. This also allows efficiently diagnosing a predisposition for a disease because suitable references can be identified in the database once it has been confirmed in the future that the subject from which the corresponding reference sample was obtained indeed developed diabetes. Preferred references which are associated with diabetes or predisposition therefor in humans are those shown in the Tables of the accompanying Examples.

The term “comparing” refers to assessing whether the amount of the qualitative or quantitative determination of glyoxylate is identical to a reference or differs therefrom.

In case the reference is obtained from a subject or a group of subjects known to suffer from diabetes or to have a predisposition for diabetes, the said disease or predisposition can be diagnosed based on the degree of identity or similarity between the amount obtained from the test sample and the aforementioned reference, i.e. based on an identical or similar qualitative or quantitative composition with respect to glyoxylate. The amount of the test sample and the reference are identical, if the values for the characteristic features and, in the case of quantitative determination, the intensity values are identical for glyoxylate. Said results are similar, if the values of the characteristic features are identical but the intensity values are different. Such a difference is, preferably, not significant and shall be characterized in that the values for the intensity are within at least the interval between 1^{st} and 99^{th} percentile, 5^{th} and 95^{th} percentile, 10^{th} and 90^{th} percentile, 20^{th} and 80^{th} percentile, 30^{th} and 70^{th} percentile, 40^{th} and 60^{th} percentile of the reference value and, most preferably, the 50^{th}, 60^{th}, 70^{th}, 80^{th}, 90^{th} or 95^{th} percentile of the reference value.

In case the reference is obtained from a subject or a group of subjects known not to suffer from diabetes or not to have a predisposition for diabetes, the said predisposition can be diagnosed based on the differences between the amount obtained from the test sample and the aforementioned reference, i.e. differences in the qualitative or quantitative composition with respect to glyoxylate. The same applies if a calculated reference as specified above is used. The difference may be an increase in the absolute or relative amount of a metabolite (sometimes referred to as up-regulation; see also Examples) or a decrease in either of said amounts or the absence of a detectable amount of the metabolite (sometimes referred to as up-regulation of the metabolite; see also Examples). Preferably, the difference in the relative or absolute amount is significant, i.e. outside of the interval between 45^{th} and 55^{th} percentile, 40^{th} and 60^{th} percentile, 30^{th} and 70^{th} percentile, 20^{th} and 80^{th} percentile, 10^{th} and 90^{th} percentile, 5^{th} and 95^{th} percentile, 1^{st} and 99^{th} percentile of the reference value. For glyoxylate, preferred values for the changes in the relative amounts (i.e. “fold”-changes) or the kind of change (i.e. “up”- or “down”-regulation resulting in a higher or lower relative and/or absolute amount) are indicated in the accompanying Tables, below. If it is indicated in said tables that glyoxylate is “up-regulated” in a subject, the relative and/or absolute amount will be increased, if it is “down-regulated”, the relative and/or absolute amount of glyoxylate will be decreased. Moreover, the “fold”-change indicates the degree of increase or decrease, e.g., a 2-fold increase means that the amount is twice the amount compared to the reference.

In a preferred embodiment of the method of the present invention, said reference is, thus, derived from a subject or a group of subjects known to suffer from diabetes or to have a predisposition therefor. More preferably, an identical or increased amount of glyoxylate in the test sample in comparison to the reference is indicative for the presence of diabetes or a predisposition therefor or wherein a decreased amount of glyoxylate in the test sample in comparison to the reference is indicative for the absence of diabetes or a predisposition therefor.

In another preferred embodiment of the method of the present invention, said reference is derived from a subject or a group of subjects known to not suffer from diabetes or to not have a predisposition therefor. More preferably, an increased amount of glyoxylate in the test sample in comparison to the reference is indicative for the presence of diabetes or a predisposition therefor or wherein an identical or decreased amount of glyoxylate in the test sample in comparison to the reference is indicative for the absence of diabetes or a predisposition therefor.

Moreover, the method of the present invention in another preferred embodiment can be used to differentiate between diabetes and a predisposition therefor and/or between different medical conditions within a subject having a predisposition for diabetes, in particular IGT or IFG. It will be understood that in such a case, the reference can be derived from a subject or a group of subjects known to exhibit IGT or IFG or a subject or a group of subjects known to suffer from diabetes. If a reference is used derived from a subject or a group of subjects known to suffer from IGT or IFG, an increase of glyoxylate shall be indicative for the presence of diabetes while an identical amount shall be indicative for the absence of diabetes and the presence of a predisposition for diabetes. If the amount of glyoxylate is, however, reduced with respect to the reference, a further comparison with a reference capable of discriminating between a healthy subject and a subject having a predisposition for diabetes shall be carried out. Suitable further references are disclosed elsewhere herein. If a reference is used derived from a subject or a group of subjects known to suffer from diabetes, a decrease of glyoxylate shall be indicative for the absence of diabetes while an identical or increased amount shall be indicative for the presence of diabetes. The diagnosis of the presence of a predisposition for diabetes, however, requires a further comparison with a reference capable of discriminating between a healthy subject and a subject having a predisposition for diabetes. Suitable further references are disclosed elsewhere herein.

The comparison is, preferably, assisted by automation. For example, a suitable computer program comprising algorithm for the comparison of two different data sets (e.g., data sets comprising the values of the characteristic feature(s)) may be used. Such computer programs and algorithm are well known in the art. Notwithstanding the above, a comparison can also be carried out manually.

As a result of the comparison carried out in step b) of the method of the present invention, thus, an aid for a final diagnosis is provided. It will be understood that a final diagnosis may require taking into account further parameters and an individual medical history of a subject. However, the method of the present invention greatly facilitates the establishment of a final diagnosis and, thus, improves diabetes care in general.

Moreover, in a preferred embodiment of the method of the present invention, said method further comprises the step of recommending based on the diagnosis established in step b) a therapy for the treatment or prevention of diabetes or a predisposition therefor.

Recommending a therapy for the treatment or prevention of diabetes or a predisposition therefor as used herein means suggesting a therapy based on the diagnostic result which may be successful in treating or preventing diabetes or which may ameliorate symptoms of diabetes for a given subject. If the diagnostic result, e.g., is the determination of the presence of diabetes, the method may comprise recommending anti-diabetes therapies or life style recommendations, such as a diet, for the subject. If the diagnostic result is, e.g., the determination of the presence of a predisposition for diabetes, the method may comprise recommending a diabetes prevention therapy or life style recommendations for preventing the development of diabetes. Suitable therapies for treating or preventing diabetes can be drug-based therapies or surgical interventions, such as bariatric surgery, and are, preferably, selected from the group consisting of: Insulin administration, incretin mimetic administration, in particular, exenatid, glucogon-like peptide 1 administration, dipeptidylpeptidase 4 (DPP IV) inhibitor administration, in particular sitagliptin, glibenclamide or glimepirid administration, glitazone administration, in particular rosiglitazone or pioglitozone, acarbose administration, glinide administration, glucoseidase inhibitor administration, metformin administration, fenretinid administration, and bariatric surgery, in particular gastric bypass surgery based on Roux en-Y procedures. Suitable therapies may also be life style recommendations such as diets, physical exercise recommendations and the like.

Preferably, it is envisaged that the said recommendation is provided automatically. This can be achieved, preferably, by providing a database comprising the recommendations allocated to different diagnostic results. The database can then be searched by a give diagnosis, i.e. a diagnostic result established in step b) of the method of the invention, for matches. The recommendations which are subsequently provided for the given diagnostic result are those which are allocated to the matching diagnostic result in the database. Such a database query system can be used as an automated expert system which provides supportive information in accordance with the method envisaged by the present invention. In particular, the method of the present invention can be applied in the context of companion diagnostics in order to recommend a certain type of therapy for an investigated subject, or not. Thus, the method of the present invention can be, preferably, used for determining whether a subject is susceptible to a therapy for treating or preventing diabetes as referred to elsewhere herein based on the diagnostic result established in step b). It will be understood that if the result is the determination of the presence of diabetes or a predisposition therefor, the subject is deemed to be susceptible to the said therapy for treating or preventing diabetes.

Advantageously, it has been found in accordance with the present invention that glyoxylate is a suitable biomarker for diagnosing diabetes or a predisposition for diabetes. This allows for a rapid, reliable and cost-effective diagnosis of diabetes or a predisposition for diabetes. Moreover, the method can be assisted by automation as described elsewhere in this description and, thus, allows high-throughput screening for subjects being at risk of suffering from diabetes. Surprisingly, glyoxylate can serve as a biomarker for diabetes or a predisposition for diabetes in regular blood donors that were not fasted (similar to HbA1C which a marker for diabetes independent of fasting status). Specifically, it has been found that glyoxylate unlike other biomarkers can be used particularly well for determining diabetes or a predisposition therefor in non-fasting subjects. Thereby, the method of the present invention may assist health programs for diabetes prevention and can be used to monitor success of preventive therapies for diabetes or other measures for the prevention of diabetes including nutritional diets. Moreover, combinations of glyoxylate and other metabolites referred to herein can be determined simultaneously in a time and cost effective manner by the metabolic profiling techniques described in this specification. Furthermore, the method of the present invention allows assessing the risk for a subject for being or becoming a member of a certain diabetes risk group, i.e. IFG, IGT or IFG&IGT. The reported prevalence of IFG and IGT varies widely between 5 and 26% depending on ethnic group, age and sex distribution. Both risk groups, IFG and IGT are expected to increase in the near future. For both, IFG or IGT risk groups, a 25% progressing to incident diabetes is reported within 3-5 years, with 50% remaining in their abnormal glycemic state and 25% reverting to normal glucose levels. With longer observation, the majority of individuals with IFG or IGT appear to develop diabetes. Individuals with both IFG and IGT (IFG&IGT) have approximately twice the rate of developing diabetes compared with subjects have either IFG or IGT (Nathan 2007, Diabetes Care 30(3): 753-759).

Moreover, the present invention contemplates a method for diagnosing diabetes accompanied by a co-morbidity or a predisposition therefor, said method comprising:
(a) determining the amount of glyoxylate in a test sample of a subject suspected to suffer from diabetes accompanied by a co-morbidity or to have a predisposition therefor wherein said sample has been obtained from the subject during an OGTT at about 2 hours after the onset of the test; and
(b) comparing the amount determined in step (a) to a reference, whereby diabetes accompanied by a co-morbidity or a predisposition therefor is to be diagnosed.

The term “co-morbidity” as used herein refers to disorders, diseases or symptoms accompanying diabetes. Such disorders or diseases may, e.g., be a direct or indirect cause of diabetes or may be directly or indirectly caused by diabetes. Preferred co-morbidities accompanying diabetes are cardiovascular disorders or disease, such as hypertension, or renal complications such as diabetic nephropathy. Most preferably, the co-morbidity accompanying diabetes referred to herein is hypertension, i.e. increased blood pressure. Hypertension is well known in the art and characterized by a blood pressure of more than 140/90 mmHg.

The sample shall be obtained at about 2 hours after the onset of the OGTT. The term “about” in this contest means +/−30 minutes, +/−15 minutes, +/−10 minutes or +/−5 minutes or precisely at 2 hours after the onset.

The “reference” referred to in the context with the aforementioned method is a reference which allows for determining whether a subject suffers from diabetes accompanied by a co-morbidity or is at risk therefor. Such a reference is accordingly either derived from a subject or group of subjects known to suffer from diabetes accompanied by a co-morbidity or a predisposition therefor or a subject or group of subjects known not to suffer from diabetes accompanied by a co-morbidity or a predisposition therefor.

If the reference is derived from a subject or group of subjects known to suffer from diabetes accompanied by a co-morbidity or a predisposition therefor an identical or increased amount of glyoxylate determined in the test sample when compared to the reference is indicative for the diabetes accompanied by the co-morbidity or the predisposition therefor. Moreover, a decreased amount is, preferably, indicative of a subject not suffering from diabetes accompanied by a co-morbidity or a predisposition therefor.

If the reference is derived from a subject or group of subjects known not to suffer from diabetes accompanied by a co-morbidity or a predisposition therefor an increased amount of glyoxylate is indicative for diabetes accompanied by a co-morbidity or a predisposition therefor. Moreover, an identical or decreased amount of glyoxylate determined in the test sample when compared to the reference is, preferably, indicative of a subject which does not suffer from diabetes accompanied by the co-morbidity or the predisposition therefor.

Advantageously, it has been found in the studies underlying the present invention that glyoxylate is also a biomarker indicating a co-morbidity accompanying diabetes such as hypertension or a predisposition therefor when determined in a sample of a subject, which has been obtained at about 2 hours after the onset of an OGTT. Particular high amounts of glyoxylate indicate, in such a case, the said co-morbidity or predisposition therefor. Accordingly, the aforementioned method allows for diagnosing the co-morbidity or predisposition therefor, monitoring of a subject with respect to the development of the said co-morbidity and/or determining whether a therapy is effective in preventing and/or ameliorating the said co-morbidity.

Moreover, the aforementioned method can be used for the development of drugs which affect diabetes and/or the co-morbidity since a possible successful drug candidate shall also influence the glyoxylate amounts found at about 2 hours after the onset of an OGTT in a test subject to which the drug candidate has been administered. Such a method may, therefore, be applied in the context of a clinical trial for a drug candidate.

In a preferred embodiment of the aforementioned method, said method comprises the further step of c) recommending a therapy against the co-morbidity if diabetes accompanied by a co-morbidity or a predisposition therefor is diagnosed in step b).

A therapy against the co-morbidity as referred to herein is, preferably, a therapy against hypertension. Such a therapy may be a drug-based therapy, a diet, or may be the recommendation of life-style adaptations such as the recommendation of physical exercise.

Moreover, the present invention relates to a method for identifying a therapy for treating or preventing diabetes comprising:
a) determining the amount of glyoxylate in a sample of a subject suffering from diabetes or having a predisposition therefor which has been subjected to a therapy suspected to be effective against diabetes or a predisposition therefor; and
b) comparing said amount to a reference, whereby a therapy effective against diabetes or a predisposition therefor is to be identified.

The term “therapy” as used herein refers to therapeutic measures which are capable of treating, preventing or ameliorating diabetes or the symptoms accompanying the disease. Preferably, said therapy is selected from the group consisting of: a drug-based therapy, a nutritional diet, a dietary supplement therapy, a surgery based therapy, such as bariatric surgery, supporting physical activity, life-style recommendations and combinations thereof.

It will be understood that the treatment as referred to in accordance with the aforementioned method will not be necessarily effective for all subjects to be treated. However, a treatment to be identified by the method shall at least be effective for a statistically significant portion of subjects of a population. Whether such a portion of subjects is statistically significant can be determined by techniques described elsewhere in this specification in detail.

Moreover, the term “subject” as used in accordance with the aforementioned method of the present invention refers to a subject which prior to the applied treatment suffered from diabetes and/or obesity.

The term “reference” in the context of the aforementioned method of the present invention refers to reference amounts of glyoxylate which are indicative for a successful treatment or prevention of diabetes. Such a reference is, preferably, obtained from a sample from a subject or group of subjects known to have been successfully treated or ameliorated from diabetes or the accompanying symptoms or prevented from developing diabetes. Moreover, references to be used in the context of this method of the invention are those indicative for the presence or absence of diabetes or a predisposition therefor referred to elsewhere herein. Thus, the reference may be obtained from sample of a subject or group of subjects known not to suffer from diabetes or a predisposition therefor, i.e. a healthy subject with respect to diabetes or a predisposition for diabetes, or a subject or group of subjects known not to suffer from diabetes or a predisposition therefor. The reference, also preferably, could be a calculated reference, most preferably the average or median, for the relative or absolute amount of glyoxylate in a population of individuals comprising the subject to be investigated.

In case a the reference is obtained from a subject or a group known to have been successfully treated or a group known not to suffer from diabetes or a predisposition for diabetes, an effective therapy can be identified based on the degree of identity or similarity between the determined amounts of glyoxylate obtained from the test sample and the aforementioned reference. In case a the reference is obtained from a subject or a group known not to suffer from diabetes or a predisposition for diabetes, an effective therapy can be identified based on the degree of identity or similarity between the determined amounts of glyoxylate obtained from the test sample and the aforementioned reference. In case a the reference is obtained from a subject or a group known to suffer from diabetes or to have a predisposition therefor, an effective therapy can be identified based on a decrease in glyoxylate amounts in the test sample in comparison to the aforementioned reference.

Advantageously, it has been found in the studies underlying the present invention that glyoxylate as a biomarker for diabetes or a predisposition therefor is, particularly, useful for identifying an effective therapy for diabetes treatment or prevention. The aforementioned method of the present invention can, thus, be applied in the development of, e.g., drug based therapies of diabetes in pre-clinical animal studies as well as clinical trials but also on a companion diagnostic level in order to identify an individually effective therapy for a given subject. Thanks to the present invention, diabetes therapies can be reliably and efficiently identified. Moreover, it can be even assessed on an individual basis whether a treatment will be effective, or not.

The present invention also relates to a method for monitoring diabetes therapy in a subject, comprising:
(a) determining the amount of glyoxylate in a first sample of said subject;
(b) determining the amount of glyoxylate in a second sample of said subject; and
(c) comparing the amount as determined in the first sample to the amount as determined in the second sample, whereby diabetes therapy in said subject is to be monitored.

The aforementioned method, preferably, is an in vitro method. Moreover, it may comprise steps in addition to those explicitly mentioned above. For example, further steps may relate to sample pre-treatments or evaluation of the results obtained by the method. Preferably, step (a), (b) and/or (c) may in total or in part be assisted by automation, e.g., by a suitable robotic and sensory equipment for the determination in steps (a) and (b), or a computer-implemented comparison in step (c).

The subject to be tested in accordance with the aforementioned method, preferably, suffers from diabetes, in particular from type 2 diabetes. However, it is also envisaged that the subject suffers from a predisposition for diabetes (for a definition of this term, see elsewhere).

The term “monitoring diabetes therapy” as used herein in the context of the aforementioned method, preferably, relates to assessing whether a subject responds to said therapy, or not. Accordingly, it is assessed whether a subject benefits from said therapy, or not. Preferably, a decrease of the amount of glyoxylate in the second sample as compared to the amount in the first sample shall be indicative for a subject who responds to diabetes therapy. In contrast, an increase of the amount (or an unchanged amount, in particular an essentially unchanged amount) of glyoxylate in the second sample as compared to the amount in the first sample shall be indicative for a subject who does not respond to diabetes therapy. Preferably, by carrying out the aforementioned method decisions can be made whether diabetes therapy in said subject shall be continued, stopped or amended.

Preferably, a subject responds to diabetes therapy, if said therapy improves the condition of the subject with respect to diabetes (e.g. if glycemic control is improved). Preferably, a subject does not respond to said therapy, if said therapy does not the improve the condition of the subject with respect to diabetes and/or any diabetes comorbidities. In this case, the therapy may put the subject at risk of adverse side effects without any significant benefit to said subject (thereby generating useless health care costs).

The term “diabetes therapy” in the context of the aforementioned method, preferably, includes any therapy for the treatment of diabetes or of a predisposition therefor. Preferred therapies are drug-based therapies and are, preferably, selected from the group consisting of: Insulin administration, incretin mimetic administration, in particular, exenatid, glucogon-like peptide 1 administration, dipeptidylpeptidase 4 (DPP IV) inhibitor administration, in particular sitagliptin, glibenclamide or glimepirid administration, glitazone administration, in particular rosiglitazone or pioglitozone, acarbose administration, glinide administration, glucoseidase inhibitor administration, metformin administration, and fenretinid administration. In another preferred embodiment, the diabetes therapy comprises nutritional therapy and life-style changes. A preferred life-style change is increased physical exercise. Preferred nutritional therapies are well known in the art and include reduced calorie intake and diets which are rich in nutrients but low in fat, in particular low in saturated fatty acids (as compared to unsaturated fatty acids).

In a preferred embodiment, the diabetes therapy includes administration of an insulin sensitizer. Preferred insulin sensitizers are metformin and thiazolidinediones. Metformin and thiazolidinediones are well known in the art. Metformin (IUPAC name: N,N-dimethylimidodicarbonimidic diamide) is an oral antidiabetic drug from the biguanide class. Preferred thiazolidinediones are selected from the group consisting of rosiglitazone (IUPAC name: 5-((4-(2-(methyl-2-pyridinylamino)ethoxy)phenyl)methyl)-2,4-thiazolidinedione), pioglitazone (IUPAC name: 5-((4-(2-(5-ethyl-2-pyridinyl)ethoxy)phenyl)methyl)-,(+-)2,4-thiazolidinedion), troglitazone (IUPAC name: 5-(4-((6-hydroxy-2,5,7,8-tetramethylchroman-2-yl-methoxy)benzyl)-2,4-thiazolidinedione). The most preferred thiazolidinedione is rosiglitazone.

The term “sample” has been described elsewhere herein. The definition applies accordingly. In the context of the aforementioned method, the amount of the biomarker as referred to herein shall be obtained in a first and in a second sample. Preferably, the first sample has been/is obtained before initiation of diabetes therapy, or more preferably, after initiation of diabetes therapy.

If the first sample has been obtained before initiation of diabetes therapy, it is preferred that it has been obtained shortly before said initiation. Preferably, a sample is considered to have been obtained shortly before initiation of diabetes therapy, if it has been obtained within less than one week, or, more preferably, within less than three days, or, most preferably, within less than one day before initiating diabetes therapy.

The “second sample” is particularly understood as a sample which is obtained in order to reflect a change of the level of the marker as referred to herein as compared to the first sample. Thus, the second sample, preferably, shall have been obtained after the first sample. Of course, the second sample shall have been obtained after initiation of diabetes therapy. It is to be understood that the second sample has been obtained not too early after the first sample in order to observe a sufficiently significant change to allow for monitoring diabetes therapy. Therefore, the second sample has been, preferably, obtained at least one week, or, more preferably, at least two weeks, or even more preferably, at least one month or two months, or, most preferably, at least three months after the first sample has been obtained. Also preferably, it is contemplated that the second sample has been obtained within a period of one week to three months after the first sample.

If the first sample has been obtained before initiation of diabetes therapy, the second sample has been, preferably, obtained at least one week, or, more preferably, at least two weeks, or even more preferably, at least one month or two months, or, most preferably, at least three months after initiation of diabetes therapy.

It shall be clear from the above that the determination of the amount of glyoxylate in the first sample referred to in step (a) may take place several days or weeks before the determination of the amount of glyoxylate in said second sample referred to in step (b). Therefore the steps (a), (b) and (c) of the method for monitoring diabetes need not be conduct one after the other in a limited time frame but may well be spread over a longer time period of several days, weeks or even months. Thus, it is to be understood that the aforementioned method allows for short-term, mid-term, and also for long-term monitoring depending on the interval between obtaining the two samples. Thus, the second sample may be obtained within a period of one day to two years or more after the first sample. In one preferred embodiment, the second sample has been obtained one day, or two days, in particular within a period of one to two days, after the first sample (which allows for short-term monitoring). In one another preferred embodiment, the second sample has been obtained one months, or two months, in particular within a period of one to two months, after the first sample (which allows for mid-term monitoring). In a further preferred embodiment, the second sample has been obtained six months, or twelve months, in particular within a period of six to twelve months or more, after the first sample (which allows for long-term monitoring).

It is also envisaged to assess the time course of the amount of glyoxylate in samples from the subject to be monitored. Accordingly, the aforementioned method may comprise the additional step of determining the amount of a said marker in at least one further sample from said subject (thus, in a third sample, in a fourth sample, in a fifth sample etc.) and comparing the, thus, determined amount with the amount of the marker in said first sample and/or in said second sample and/or in any sample that was obtained before said at least one further sample was obtained. For preferred time intervals for obtaining the samples, please see above.

Preferably, the assessment whether the subject responds to diabetes therapy, or not, is based on the comparison of the amount of glyoxylate in a first sample from the subject with the amount of the respective marker in a second sample from said subject.

Preferably, a decrease and, more preferably, a significant decrease, and, most preferably, a statistically significant decrease of the amount of glyoxylate in the second sample as compared to the first sample is indicative for a subject who responds to diabetes therapy.

A significant decrease, preferably, is a decrease of a size which is considered to be significant for monitoring diabetes. Particularly said decrease is considered statistically significant. The terms “significant” and “statistically significant” are known by the person skilled in the art. Thus, whether a decrease is significant or statistically significant can be determined without further ado by the person skilled in the art using various well known statistic evaluation tools. Preferred significant decreases of the amount of glyoxylate which are indicative for a subject who responds to diabetes therapy are given herein below

Preferably, a decrease of the amount of glyoxylate in the second sample compared to the amount in the first sample, preferably, of at least 5%, of at least 10%, more preferably of at least 20%, and, even more preferably, of at least 30%, and most preferably of at least 40% is considered to be significant and, thus, to be indicative for a subject who responds to diabetes therapy.

As set forth above, an increase of the amount of glyoxylate in the second sample compared with the first sample (or an, in particular, an essentially unchanged amount of the amount of glyoxylate in the second sample compared with the first sample) is indicative for a subject who does not respond to diabetes therapy.

In a preferred embodiment the aforementioned method further comprises the steps of (a1) comparing the amount determined in the first sample to a reference amount, and of (b1) comparing the amount determined in the second sample to a reference amount.

The present invention also pertains to a method for monitoring diabetes therapy in a subject, comprising:
(a) determining the amount of glyoxylate in a first sample of said subject;
(a1) comparing the amount determined in step (a) to a reference,
(b) determining the amount of glyoxylate in a second sample of said subject;
(b1) comparing the amount determined in step (b) to a reference, and
(c) comparing the amount as determined in the first sample to the amount as determined in the second sample, whereby diabetes therapy in said subject is to be monitored.

A suitable reference for the comparison carried out in steps (a1) and (b1), preferably, is a reference as specified elsewhere herein. In a preferred embodiment the reference may be derived from a healthy subject. Preferably, however, the reference is derived from a subject suffering from diabetes or having a predisposition therefor. The comparison carried out in steps (a1) and (b1) provide further diagnostic information. E.g., by carrying out the further steps, the severity of diabetes may be assessed.

The explanations and interpretations of the terms made above, preferably, apply accordingly to the other embodiments specified herein below.

In general, the present invention relates to the use in a sample of a subject of glyoxylate or a detection agent for glyoxylate for diagnosing diabetes or a predisposition therefor. Preferably, the detection agent is an antibody which specifically binds to glyoxylate or an aptamere which specifically binds to glyoxylate as specified elsewhere herein in detail.

Further, the present invention, in general, contemplates the use in a sample of a subject of glyoxylate or a detection agent for glyoxylate for identifying a subject being susceptible to a therapy for treating or preventing diabetes. Preferably, the detection agent is an antibody which specifically binds to glyoxylate or an aptamere which specifically binds to glyoxylate as specified elsewhere herein in detail.

Further, the present invention, in general, contemplates the use of glyoxylate or a detection agent for glyoxylate in a first and second sample of a subject for monitoring diabetes therapy. Preferably, the detection agent is an antibody which specifically binds to glyoxylate or an aptamere which specifically binds to glyoxylate as specified elsewhere herein in detail.

The present invention also relates to a device for diagnosing diabetes or a predisposition therefor in a sample of a subject suspected to suffer therefrom comprising:
(a) an analyzing unit comprising a detection agent for glyoxylate which allows for determining the amount of glyoxylate present in the sample; and, operatively linked thereto,
(b) an evaluation unit comprising a stored reference and a data processor which allows for comparing the amount of glyoxylate determined by the analyzing unit to the stored reference, whereby diabetes or the predisposition therefor is diagnosed.

The methods of the present invention can be implemented by the aforementioned device. A device as used herein shall comprise at least the aforementioned units. The units of the device are operatively linked to each other. How to link the units in an operating manner will depend on the type of units included into the device. For example, where means for automatically qualitatively or quantitatively determining glyoxylate are applied in an analyzing unit, the data obtained by said automatically operating unit can be processed by the evaluation unit, e.g., by a computer program which runs on a computer being the data processor in order to facilitate the diagnosis. Preferably, the units are comprised by a single device in such a case. However, the analyzing unit and the evaluation unit may also be physically separate. In such a case operative linkage can be achieved via wire and wireless connections between the units which allow for data transfer. A wireless connection may use Wireless LAN (WLAN) or the internet. Wire connections may be achieved by optical and non-optical cable connections between the units. The cables used for wire connections are, preferably, suitable for high throughput data transport

A preferred analyzing unit for determining glyoxylate comprises a detection agent, such as an antibody, protein or aptamere which specifically recognizes glyoxylate as specified elsewhere herein, and a zone for contacting said detection agent with the sample to be tested. The detection agent may be immobilized on the zone for contacting or may be applied to said zone after the sample has been loaded. The analyzing unit shall be, preferably, adapted for qualitatively and/or quantitatively determine the amount of complexes of the detection agent and glyoxylate. It will be understood that upon binding of the detection agent to the glyoxylate, at least one measurable physical or chemical property of either glyoxylate, the detection agent or both will be altered such that the said alteration can be measured by a detector, preferably, comprised in the analyzing unit. However, where analyzing units such as test stripes are used, the detector and the analyzing units may be separate components which are brought together only for the measurement. Based on the detected alteration in the at least one measurable physical or chemical property, the analyzing unit may calculate an intensity value for glyoxylate as specified elsewhere herein. Said intensity value can then be transferred for further processing and evaluation to the evaluation unit. Alternatively, an analyzing unit as referred to herein, preferably, comprises means for separating metabolites, such as chromatographic devices, and means for metabolite determination, such as spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS and/or GC-MS is used in the analyzing unit referred to in accordance with the present invention.

The evaluation unit of the device of the present invention, preferably, comprises a data processing device or computer which is adapted to execute rules for carrying out the comparison as specified elsewhere herein. Moreover, the evaluation unit, preferably, comprises a database with stored references. A database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for diabetes or a predisposition thereof (e.g. a query search). Thus, if an identical or similar data set can be identified in the data collection, the test data set will be associated with diabetes or a predisposition for diabetes. The evaluation unit may also preferably comprise or be operatively linked to a further database with recommendations for therapeutic or preventive interventions or life style adaptations based on the established diagnosis of diabetes or a predisposition therefor. Said further database can be, preferably, automatically searched with the diagnostic result obtained by the evaluation unit in order to identify suitable recommendations for the subject from which the test sample has been obtained in order to treat or prevent diabetes.

In a preferred embodiment of the device of the present invention, said stored reference is a reference derived from a subject or a group of subjects known to suffer from diabetes or to have a predisposition therefor and said data processor executes instructions for comparing the amount of glyoxylate determined by the analyzing unit to the stored reference, wherein an identical or increased amount of glyoxylate in the test sample in comparison to the reference is indicative for the presence of diabetes or a predisposition therefor or wherein a decreased amount of glyoxylate in the test sample in comparison to the reference is indicative for the absence of diabetes or a predisposition therefor.

In another preferred embodiment of the device of the present invention, said stored reference is a reference derived from a subject or a group of subjects known to not suffer from diabetes or to not have a predisposition therefor and said data processor executes instructions for comparing the amount of glyoxylate determined by the analyzing unit to the stored reference, wherein an increased amount of glyoxylate in the test sample in comparison to the reference is indicative for the presence of diabetes or a predisposition therefor or wherein an identical or decreased amount of glyoxylate in the test sample in comparison to the reference is indicative for the absence of diabetes or a predisposition therefor.

Advantageously, the device of the present invention allows for automated diagnosis of diabetes or a predisposition for diabetes. The device, thus, can also be used without special medical knowledge by medicinal staff or patients, in particular when an expert system making recommendations as specified above is included. The device is also suitable for near-patient applications since the device can be adapted to a portable format.

The present invention also encompasses a kit for diagnosing diabetes or a predisposition therefor comprising a detection agent for glyoxylate and preferably also glyoxylate standards in an concentration derived from a subject or a group of subjects known to suffer from diabetes or to have a predisposition therefor or derived from a subject or a group of subjects known to not suffer from diabetes or to not have a predisposition therefor. Preferably, the detection agent is an antibody which specifically binds to glyoxylate or an aptamere which specifically binds to glyoxylate as specified elsewhere herein in detail.

A “standard” as referred to herein is an amount of glyoxylate when present in solution or dissolved in a predefined volume of a solution resembles the amount of glyoxylate which is present in a subject or a group of subjects known to suffer from diabetes or to have a predisposition therefor or derived from a subject or a group of subjects known to not suffer from diabetes or to not have a predisposition therefor.

The term “kit” refers to a collection of the aforementioned components, preferably, provided separately or within a single container. The container also comprises instructions for carrying out the method of the present invention. These instructions may be in the form of a manual or may be provided by a computer program code which is capable of carrying out the comparisons referred to in the methods of the present invention and to establish a diagnosis accordingly when implemented on a computer or a data processing device. The computer program code may be provided on a data storage medium or device such as an optical or magnetic storage medium (e.g., a Compact Disc (CD), CD-ROM, a hard disk, optical storage media, or a diskette) or directly on a computer or data processing device.

All references referred to above are herewith incorporated by reference with respect to their entire disclosure content as well as their specific disclosure content explicitly referred to in the above description.

### EXAMPLES

The invention will now be illustrated by the following Examples which are not intended to restrict or limit the scope of this invention.

### Example 1

### General Study Goal and Design

To identify such a biomarker or combination of biomarkers, a diabetes screening study was designed selected from a cohort of 87033 regular and long-term blood donors at the blood bank of the Bavarian Red Cross. The study was subdivided into two parts. The prospective diabetes screening part included an oral glucose tolerance test (OGTT) assessment (prospective part) for categorization of study participants and metabolic differentiation between diabetes categories based on fasting plasma glucose (FPG) and findings of the OGTT. The retrospective part allowed evaluating early metabolic changes in the course of developing diabetes up to six years prior to diagnosis. Of the total blood donor cohort 60859 participated in the diabetes screening study and 60656 of those completed a diabetes risk score assessment termed “Findrisk” (Martin 2007, Dtsch Med Wochenschr. 132(24): 1315-1320). Of those participants 16.1% showed an elevated risk for diabetes ≧12. Of those participants with elevated risk for diabetes based on the Findrisk score a total of 4241 blood donors were identified with the additional risk of developing elevated long-term blood glucose levels as indicated by hemoglobin A1C (HbA1C) values of ≧5.6%. The study also included subjects with Findrisk score below 12 and with HbA1C values<5.6% that served as controls.

### Example 2

### Prospective Study

Subjects were selected for the prospective study part from a total of 789 participants that volunteered to participate in the OGTT assessment. Prior to selection participants were grouped according to their fasting plasma glucose (prior to the OGTT) and according to their OGTT categorization.

Standard WHO diabetes categories were applied (WHO 2006):

Diabetes: FPG≧7.0 mmol/L or 2HPG≧11.1 mmol/L;

IGT: FPG<7.0 mmol/L and 2HPG≧7.8 and <11.1 mmol/L;

IFG: FPG 6.1 to 6.9 mmol/L and 2HPG<7.8 mmol/L,

Healthy: FPG≦5 6.0 mmol/L and 2HPG<7.8 mmol/L).

2HPG=plasma 2 h after standardized 75 g oral glucose challenge

Selection was performed for best matching of the diabetes categories as well as potential confounders such as center, gender, body mass index and age. Finally, 478 study participants were included into the prospective study part.

TABLE 1A
Diabetes categories after measurements of fasting
plasma glucose and OGTT assessment for
the 478 participants in the prospective study part

| Diabetics | Diabetics | | | | |
|---|---|---|---|---|---|
| identified only | identified only | | | | |
| through 2HPG | or additionally | | | | |
| not through FPG | through FPG | IFG + IGT | IGT | IFG | Healthy |
| 28 | 30 | 77 | 39 | 127 | 177 |

TABLE 1B
Subset of above described subjects measured with the
SIM method at OGTT 120

| Diabetics | Diabetics | | | | |
|---|---|---|---|---|---|
| identified only | identified only | | | | |
| through 2HPG | or additionally | | | | |
| not through FPG | through FPG | IFG + IGT | IGT | IFG | Healthy |
| 23 | 23 | 55 | 36 | 98 | 50 |

Metabolite profiling was performed on the fasted plasma samples obtained from study participants directly prior to the OGTT, as well as on plasma samples 120 min after standard oral glucose bolus (75 g). Plasma was processed by standard protocols and separated from blood within approximately 60 min. Plasma samples were immediately frozen and stored at −80° C. Transport of samples from sampling site to site of biochemical analysis was on dry ice.

### Example 3

### Retrospective Study

The retrospective study part was performed on long-term storage archive samples from the study participants that were categorized into diabetes categories for the prospective study part based on fasting plasma and OGTT glucose levels. Four retrospective samples per subject were obtained from the controlled storage facility of the Bavarian Red Cross. The four samples included a plasma sample from (1) the last regular blood donation prior to OGTT and typically a plasma sample from (2) 18 months prior to the last donation, (3) 36 months prior to the last donation and (4) 72 months prior to the last donation. All samples for the retrospective study part were sampled, processed and stored according to strict standard operating procedures of the blood bank.

TABLE 2A
Diabetes categories after measurements of fasting plasma
glucose and OGTT assessment for the 243 participants
in the retrospective study part

| Diabetics | Diabetics | | | | |
|---|---|---|---|---|---|
| identified only | identified only | | | | |
| through 2HPG | or additionally | | | | |
| not through FPG | through FPG | IFG + IGT | IGT | IFG | Healthy |
| 27 | 28 | 50 | 10 | 32 | 96 |

TABLE 2B
Subset of above described subjects measured with the SIM method

| Diabetics identified only through | Diabetics identified only or | |
|---|---|---|
| 2HPG not through FPG | additionally through FPG | Healthy |
| 24 | 23 | 51 |

Metabolite profiling was performed on plasma samples of regular blood donors that later become study participants. Prior to the blood donation donors were encouraged to eat. Plasma was separated immediately after blood donation. Plasma samples were then stored at approximately 4° C. for about 24 h until preparation of aliquots and transfer into long-term storage at −42° C. Transport of samples from long-term storage to site of biochemical analysis was performed on dry ice.

### Example 4

### Analysis of Plasma Samples from the Studies

Prospective and retrospective plasma samples were categorized into discrete diabetes risk groups according to results from FPG and OGTT and subsequently analyzed by broad profiling and metabolomic characterization. Samples were prepared and subjected to LC-MS/MS, GC-MS and SPE-LC-MS/MS (hormones) analysis as described below. Several groups of metabolites were analyzed semi-quantitatively or quantitatively including amino acids, carbohydrates, fatty acids, mono-, di- and triglycerides, other lipids, organic acids, coenzymes, vitamins, secondary metabolites, steroid hormones and catecholamines. Prospective samples were also analyzed for selected eicosanoids.

Proteins were separated by precipitation from blood plasma. After addition of water and a mixture of ethanol and dichlormethan the remaining sample was fractioned into an aqueous, polar phase and an organic, lipophilic phase.

For the transmethanolysis of the lipid extracts (lipophilic phase) a mixture of 140 μl of chloroform, 37 μl of hydrochloric acid (37% by weight HCl in water), 320 μl of methanol and 20 μl of toluene was added to the evaporated extract. The vessel was sealed tightly and heated for 2 hours at 100° C., with shaking. The solution was subsequently evaporated to dryness. The residue was dried completely.

The methoximation of the carbonyl groups was carried out by reaction with methoxyamine hydrochloride (20 mg/ml in pyridine, 100 μl for 1.5 hours at 60° C.) in a tightly sealed vessel. 20 μl of a solution of odd-numbered, straight-chain fatty acids (solution of each 0.3 mg/mL of fatty acids from 7 to 25 carbon atoms and each 0.6 mg/mL of fatty acids with 27, 29 and 31 carbon atoms in 3/7 (v/v) pyridine/toluene) were added as time standards. Finally, the derivatization with 100 μl of N-methyl-N-(trimethylsilyl)-2,2,2-trifluoroacetamide (MSTFA) was carried out for 30 minutes at 60° C., again in the tightly sealed vessel. The final volume before injection into the GC was 220 μl.

For the polar phase the derivatization was performed in the following way: The methoximation of the carbonyl groups was carried out by reaction with methoxyamine hydrochloride (20 mg/ml in pyridine, 50 μl for 1.5 hours at 60° C.) in a tightly sealed vessel. 10 μl of a solution of odd-numbered, straight-chain fatty acids (solution of each 0.3 mg/mL of fatty acids from 7 to 25 carbon atoms and each 0.6 mg/mL of fatty acids with 27, 29 and 31 carbon atoms in 3/7 (v/v) pyridine/toluene) were added as time standards. Finally, the derivatization with 50 μl of N-methyl-N-(trimethylsilyl)-2,2,2-trifluoroacetamide (MSTFA) was carried out for 30 minutes at 60° C., again in the tightly sealed vessel. The final volume before injection into the GC was 110 μl. The GC-MS systems consist of an Agilent 6890 GC coupled to an Agilent 5973 MSD. Autosamplers were CompiPal or GCPal from CTC.

For the analysis usual commercial capillary separation columns (30 m×0.25 mm×0.25 μm) with different poly-methyl-siloxane stationary phases containing 0% to 35% of aromatic moieties, depending on the analyzed sample materials and fractions from the phase separation step, were used (for example: DB-1 ms, HP-5 ms, DB-XLB, DB-35 ms, Agilent Technologies). Up to 1 μL of the final volume was injected splitless and the oven temperature program was started at 70° C. and ended at 340° C. with different heating rates depending on the sample material and fraction from the phase separation step in order to achieve a sufficient chromatographic separation and number of scans within each analyte peak. Furthermore RTL (Retention Time Locking, Agilent Technologies) was used for the analysis and usual GC-MS standard conditions, for example constant flow with nominal 1 to 1.7 ml/min. and helium as the mobile phase gas, ionisation was done by electron impact with 70 eV, scanning within a m/z range from 15 to 600 with scan rates from 2.5 to 3 scans/sec and standard tune conditions.

The HPLC-MS systems consisted of an Agilent 1100 LC system (Agilent Technologies, Waldbronn, Germany) coupled with an API 4000 Mass spectrometer (Applied Biosystem/MDS SCIEX, Toronto, Canada). HPLC analysis was performed on commercially available reversed phase separation columns with C18 stationary phases (for example: GROM ODS 7 pH, Thermo Betasil C18). Up to 10 μL of the final sample volume of evaporated and reconstituted polar and lipophilic phase was injected and separation was performed with gradient elution using methanol/water/formic acid or acetonitrile/water/formic acid gradients at a flowrate of 200 μL/min. Mass spectrometry was carried out by electrospray ionisation in positive mode for the non-polar fraction and negative mode for the polar fraction using multiple-reaction-monitoring-(MRM)-mode and fullscan from 100-1000 amu.

### Example 5

### Data Analysis and Statistical Evaluation

Plasma samples were analyzed in randomized analytical sequence design with pooled samples (so called “pool”) generated from aliquots of each sample. Following comprehensive analytical validation steps, the raw peak data for each analyte were normalized to the median of pool per analytical sequence to account for process variability (so called “pool-normalized ratios”). If available, absolute concentrations of metabolites were used for statistical analysis. In all other cases, pool-normalized ratios were used.

All data were log 10-transformed to achieve about normal distribution. Mixed-effects models were computed correcting data for confounders (sample storage time, center, gender, body mass index (BMI), subject age) and estimating diabetes diagnostics results obtained from oral glucose tolerance test (OGTT). In addition to FPG results were obtained during the OGTT at the 120 min post glucose load time point. Subjects were differentiated based on previous anti-hypertensive treatment (treatment against high blood pressure) and profiling patterns compared between these subgroups. Biomarker importance was read out from statistically significant p-values of t-statistics. Direction and strength of regulation were obtained in better human readable format by transforming back estimated effects from log 10-ratio-scale to multiplicative ratio scale. In order to identify early biomarkers of diabetes or diabetes risk, effects were read out for all available time points up to six years before diabetes diagnostics OGTT results.

The results for glyoxylate as a biomarker identified in the studies described above are summarized in the following tables, below.

TABLE 3
Performance of glyoxylate as predictor for diabetes or diabetes risk in the prospective
dataset; comparisons were made from fasted plasma samples prior to the OGTT among several
diabetes risk groups. p-values correspond to t-statistics of confounder-correcting fixed-effects
ANOVA models Ratios correspond to effects estimated on logratio scale transformed to
multiplicative ratio scale (ratio = 10 \hat{ } (estimated effect on logratio scale)). Direction corresponds
to direction of regulation in positive subjects (diabetes and/or risk subjects) vs. negative
subjects (healthy controls).

| Diagnostic question | Direction | p-value | Ratio |
|---|---|---|---|
| Diabetes vs. healthy | Up | 0.011 | 1.23 |
| Non-healthy (risk & diabetes) vs. healthy subjects | Up | 0.017 | 1.13 |
| Risk by OGTT vs. healthy | Up | 0.024 | 1.16 |
| All risk subjects vs. healthy | Up | 0.052 | 1.11 |
| Glucose-based comparison positive (diabetes and risk | Up | 0.0026 | 1.19 |
| subjects) vs. neg negative (healthy controls, (quantile | | | |
| thresholds with gap in glucose concentration) | | | |
| Correlation with numeric HbA1c | Up | 0.056 | 1.05¹ |
| HbA1c-based comparison pos vs. neg (standard | Up | 0.036 | 1.15 |
| thresholds with gap) | | | |
| HbA1c-based comparison pos vs. neg (quantile | Up | 0.039 | 1.13 |
| thresholds with gap) | | | |
| Diabetes vs. IFG | Up | 0.095 | 1.15 |
| IFG + IGT vs. healthy | Up | 0.0043 | 1.23 |
| Diabetes vs. IGT | Up | 0.095 | 1.20 |
| Diabetes detectable by fasting glucose vs. healthy | Up | 0.0082 | 1.32 |

¹Ratio corresponds to estimated change of Glyoxylate by one standard deviation change of HbA1c in the current dataset (sd(HbA1c) = 0.34).

The performance of glyoxylate as predictor for diabetes or diabetes risk in the prospective data-set (see table above) can be improved by combining measurement and analysis of glyoxylate with known diabetes markers such as glucose, HbA1C, 1,5 Anhydrosorbitol, 2-Hydroxybutyrate or Mannose.

The following comparisons were made over time in the diabetes risk groups (temporal comparisons):
A) Diabetes or risk group vs. healthy subjects for median time point 2.6 years (y) prior to last blood donation (linear interpolation between time points)
B) Diabetes or risk group vs. healthy subjects at time point 0 y (last blood donation)
C) Diabetes or risk group vs. healthy subjects 1.5 years prior to last blood donation)
D) Diabetes or risk group vs. healthy subjects 3 years prior to last blood donation)
E) Diabetes or risk group vs. healthy subjects 6 years prior to last blood donation)
F) Deviation of the linear slopes comparing slopes from diabetes or risk groups vs. healthy subjects (interactions between ANOVA factors diabetes status and time)

TABLE 4
Performance of glyoxylate as predictor for diabetes or diabetes risk in the retrospective
dataset; comparisons were made over time in the diabetes risk groups. . p-values correspond to
t-statistics of confounder-correcting fixed-effects ANOVA models. Ratios correspond to effects
estimated on logratio scale transformed to multiplicative ratio scale (ratio = 10 \hat{ } (estimated
effect on logratio scale)). Direction corresponds to direction of regulation in positive subjects
(diabetes and/or risk subjects) vs. negative subjects (healthy controls). Direction of regulation
compares diabetics or risk groups to healthy control subjects at a significance level of
p-value < 0.05.

| Diagnostic question | Direction | p-value | Ratio |
|---|---|---|---|
| Diabetes vs. healthy subjects for median time point | Up | 0.0027 | 1.21 |
| 2.6y prior to last blood donation (linear interpolation | | | |
| between time points) | | | |
| Diabetes vs. healthy subjects at time point 0y (last | Up | 0.0015 | 1.44 |
| blood donation) | | | |
| Diabetes vs. healthy subjects 3years prior to last | Up | 0.0084 | 1.35 |
| blood donation | | | |
| lincrease of Glyoxylate over time difference between | Up | 0.0093 | 1.16¹ |
| diabetes and healthy subjects (comparison of slopes | | | |
| for linear time estimation) | | | |
| Impaired glucose tolerance vs. healthy subjects for | Up | 0.026 | 1.51 |
| median time point 2.6y prior to last blood donation | | | |
| (linear interpolation between time points) | | | |
| Impaired glucose tolerance vs. healthy subjects 3 | Up | 0.0036 | 2.00 |
| years prior to last blood donation | | | |
| Subjects from diabetes and all risk groups vs. | Up | 0.0039 | 1.15 |
| healthy subjects for median time point 2.6y prior to | | | |
| last blood donation (linear interpolation between | | | |
| time points) | | | |
| Subjects from diabetes and all risk groups healthy | Up | 0.036 | 1.21 |
| subjects at time point 0y (last blood donation) | | | |
| Subjects from diabetes and all risk groups vs. | Up | 0.034 | 1.21 |
| healthy subjects 3 years prior to last blood donation | | | |

¹Ratio corresponds to estimated change of Glyoxylate by one standard deviation change on time scale with equidistant steps time = 0, 1, 2, 3 for 0y, 1.5y, 3y, 6y in the current dataset.

TABLE 5
Correlation of glyoxylate with numeric HbA1C measurements; comparisons were made
over time in diabetes risk groups at a significance level of p-values <0.05. p-values correspond
to t-statistics of confounder-correcting fixed-effects ANOVA models. Ratios correspond to
effects estimated on logratio scale transformed to multiplicative ratio scale (ratio = 10 \hat{ }
(estimated effect on logratio scale)).

| Diabetes risk comparison | p-value | Ratio |
|---|---|---|
| Correlation with numeric HbA1c; 2.6y; males & females | 0.011 | 1.06 |
| Correlation with numeric HbA1c; 2.6y; males | 0.022 | 1.06 |
| Correlation with numeric HbA1c; 0y; males & females | 0.013 | 1.12 |
| Correlation with numeric HbA1c; 3y; males & females | 0.016 | 1.11 |
| Increase of the correlation between Glyoxylate and HbA1c over | 0.026 | 1.05 |
| time (Interaction of the two numeric factors HbA1c and time, both | | |
| factors standardized for ANOVA) | | |

TABLE 6
Performance of glyoxylate as predictor for increased blood pressure associated
diabetes complication 120 min after oral glucose challenge in the prospective dataset measured
by SIM. p-values correspond to t-statistics of confounder-correcting fixed-effects ANOVA
models. Ratios correspond to effects estimated on logratio scale transformed to multiplicative ratio
scale (ratio = 10 \hat{ } (estimated effect on logratio scale)). Direction corresponds to direction of
regulation in positive subjects (diabetes and/or risk subjects) vs. negative subjects
(healthy controls).

| Diagnostic question | Direction | p-value | Ratio |
|---|---|---|---|
| Difference between subjects with and without previous | Up | 0.0043 | 1.72 |
| anti-hypertensive treatment in the comparison of | | | |
| diabetes vs. healthy at the 120 min OGTT time point. | | | |

TABLE 7
Performance of glyoxylate as predictor for diabetes or diabetes risk diagnosed by
OGTT in the retrospective dataset after SIM measurement; comparisons were made over time
in the diabetes risk groups. p-values correspond to t-statistics of confounder-correcting fixed-
effects ANOVA models. Ratios correspond to effects estimated on logratio scale transformed to
multiplicative ratio scale (ratio = 10 \hat{ } (estimated effect on logratio scale)). Direction
corresponds to direction of regulation in positive subjects (diabetes and/or risk subjects) vs.
negative subjects (healthy controls). Direction of regulation compares diabetics or risk groups
to healthy control subjects at a significance level of p-value < 0.05.

| Diagnostic question | Direction | p-value | Ratio |
|---|---|---|---|
| Diabetes classified only by OGTT (not by elevated | Up | 0.0407 | 1.1096 |
| fasting glucose) vs. healthy subjects for median time | | | |
| point 2.6y prior to last blood donation (linear | | | |
| interpolation between time points) | | | |
| Diabetes classified only by OGTT (not by elevated | Up | 0.0139 | 1.2097 |
| fasting glucose) vs. healthy subjects at time point 0y | | | |
| (last blood donation) | | | |
| Diabetes classified only by OGTT (not by elevated | Up | 0.0463 | 1.1665 |
| fasting glucose) vs. healthy subjects 6 years prior to | | | |
| last blood donation | | | |

### Example 6

### SIM Method Description

Prospective and retrospective plasma samples were categorized into discrete diabetes risk groups according to results from FPG and OGTT and subsequently analyzed by broad profiling and metabolomic characterization. Samples were prepared and subjected to LC-MS/MS, GC-MS and SPE-LC-MS/MS (hormones) analysis as described below. Several groups of metabolites were analyzed semi-quantitatively or quantitatively including amino acids, carbohydrates, fatty acids, mono-, di-and triglycerides, other lipids, organic acids, coenzymes, vitamins, secondary metabolites, steroid hormones and catecholamines.

Proteins were separated by precipitation from blood plasma. After addition of water and a mixture of ethanol and dichlormethan the remaining sample was fractioned into an aqueous, polar phase and an organic, lipophilic phase.

For the transmethanolysis of the lipid extracts (lipophilic phase) a mixture of 140 μl of chloroform, 37 μl of hydrochloric acid (37% by weight HCl in water), 320 μl of methanol and 20 μl of toluene was added to the evaporated extract. The vessel was sealed tightly and heated for 2 hours at 100° C., with shaking. The solution was subsequently evaporated to dryness. The residue was dried completely.

The methoximation of the carbonyl groups was carried out by reaction with methoxyamine hydrochloride (20 mg/ml in pyridine, 100 l for 1.5 hours at 60° C.) in a tightly sealed vessel. 20 μl of a solution of odd-numbered, straight-chain fatty acids (solution of each 0.3 mg/mL of fatty acids from 7 to 25 carbon atoms and each 0.6 mg/mL of fatty acids with 27, 29 and 31 carbon atoms in 3/7 (v/v) pyridine/toluene) were added as time standards. Finally, the derivatization with 100 μl of N-methyl-N-(trimethylsilyl)-2,2,2-trifluoroacetamide (MSTFA) was carried out for 30 minutes at 60° C., again in the tightly sealed vessel. The final volume before injection into the GC was 100 μl.

For the polar phase the derivatization was performed in the following way: The methoximation of the carbonyl groups was carried out by reaction with methoxyamine hydrochloride (20 mg/ml in pyridine, 50 l for 1.5 hours at 60° C.) in a tightly sealed vessel. 10 μl of a solution of odd-numbered, straight-chain fatty acids (solution of each 0.3 mg/mL of fatty acids from 7 to 25 carbon atoms and each 0.6 mg/mL of fatty acids with 27, 29 and 31 carbon atoms in 3/7 (v/v) pyridine/toluene) were added as time standards. Finally, the derivatization with 50 μl of N-methyl-N-(trimethylsilyl)-2,2,2-trifluoroacetamide (MSTFA) was carried out for 30 minutes at 60° C., again in the tightly sealed vessel. The final volume before injection into the GC was 100 μl. The GC-MS systems consist of an Agilent 6890 GC coupled to an Agilent 5973 MSD. Autosamplers were CompiPal or GCPal from CTC.

For the analysis usual commercial capillary separation columns (30 m×0.25 mm×0.25 μm) with different poly-methyl-siloxane stationary phases containing 0% to 35% of aromatic moieties, depending on the analyzed sample materials and fractions from the phase separation step, were used (for example: DB-1 ms, HP-5 ms, DB-XLB, DB-35 ms, Agilent Technologies). Up to 1 μL of the final volume was injected splitless and the oven temperature program was started at 70° C. and ended at 340° C. with different heating rates depending on the sample material and fraction from the phase separation step in order to achieve a sufficient chromatographic separation and number of scans within each analyte peak. Furthermore RTL (Retention Time Locking, Agilent Technologies) was used for the analysis and usual GC-MS standard conditions, for example constant flow with nominal 1 to 1.7 ml/min. and helium as the mobile phase gas and standard tune conditions were applied. Ionisation was done by electron impact with 70 eV, scanning 2-3 characteristic mass fragments of each analyte within an appropriate time window that consisted of 2 to 13 ion masses. Scan rates ranged from 3 to 14 scans/sec depending on the number of scanned masses within the respective time windows.

### Example 7

### Rats Treated with Anti-Diabetic Drug Metformin

Two groups of each 5 male and female rats was dosed once daily with the indicated compounds with a different dose per group (see below) over 28 days.

Each dose group in the studies consisted of five rats per sex. Additional groups of each 15 male and 15 female animals served as controls. Before starting the treatment period, animals, which were 62-64 days old when supplied, were acclimatized to the housing and environmental conditions for 7 days. All animals of the animal population were kept under the same constant temperature (20-24±3° C.) and the same constant humidity (30-70%). The animals of the animal population were fed ad libitum. The food to be used was essentially free of chemical or microbial contaminants. Drinking water was also offered ad libitum. Accordingly, the water was free of chemical and microbial contaminants as laid down in the European Drinking Water Directive 98/83/EG. The illumination period was 12 hours light followed by 12 hours darkness (12 hours light, from 6:00 to 18:00, and 12 hours darkness, from 18:00 to 6:00). The studies were performed in an AAALAC-approved laboratory in accordance with the German Animal Welfare Act and the European Council Directive 86/609/EE. The test system was arranged according to the OECD 407 guideline for the testing of chemicals for repeated dose 28-day oral toxicity study in rodents. The test substance was dosed and administered as follows:

Metformin hydrochloride was administered by gavage (high dose group at 1 g/kg body weight, low dose group at 0.2 g/kg body weight), in drinking water containing 0.5% Carboxymethyl cellulose (Tylose CB30000) (administration volume: 10 ml/kg body weight).

In the morning of day 7, 14, and 28, blood was taken from the retroorbital venous plexus from fasted anaesthetized animals. From each animal, 1 ml of blood was collected with EDTA as anticoagulant. The samples were centrifuged for generation of plasma. All plasma samples were covered with a N₂ atmosphere and then stored at −80° C. until analysis.

For mass spectrometry-based metabolite profiling analyses plasma samples were extracted and a polar and a non-polar (lipid) fraction was obtained. For GC-MS analysis, the non-polar fraction was treated with methanol under acidic conditions to yield the fatty acid methyl esters. Both fractions were further derivatised with O-methyl-hydroxyamine hydrochloride and pyridine to convert Oxo-groups to O-methyloximes and subsequently with a silylating agent before analysis. In LC-MS analysis, both fractions were reconstituted in appropriate solvent mixtures. HPLC was performed by gradient elution on reversed phase separation columns. Mass spectrometric detection which allows target and high sensitivity MRM (Multiple Reaction Monitoring) profiling in parallel to a full screen analysis was applied as described in WO2003073464.

Steroids and their metabolites were measured by online SPE-LC-MS (Solid phase extraction-LC-MS). Catecholamines and their metabolites were measured by online SPE-LC-MS as described by Yamada et al. (Yamada 2002, Journal of Analytical Toxicology, 26(1): 17-22)

Following comprehensive analytical validation steps, the data for each analyte were normalized against data from pool samples. These samples were run in parallel through the whole process to account for process variability. The significance of treatment group values specific for sex, treatment duration and metabolite was determined by comparing means of the treated groups to the means of the respective untreated control groups using WELCH-test and quantified with treatment ratios versus control and p-values.

TABLE 8
Effects of metformin on healthy rat plasma glyoxylate concentrations. F7, f14 and f28
refer to rat plasma taken from female rats 7, 14 and 28 days after the start of dosing respective-
ly. Likewise m7, m14 and m28 refer to rat plasma taken from male rats 7, 14 and 28 days after
the start of dosing respectively.

| | Metformin hydrochloride | | | | | |
|---|---|---|---|---|---|---|
| | f7 | f14 | f28 | m7 | m14 | m28 |
| High Dose | | | | | | |
| Ratio treat- | 0.82 | 0.58 | 0.52 | 0.22 | 0.29 | 0.58 |
| ment/control | | | | | | |
| P-value | 0.24 | 0.01 | 0.08 | 0.00 | 0.00 | 0.10 |
| Low Dose | | | | | | |
| Ratio treat- | 1.01 | 0.72 | 0.64 | 0.59 | 0.65 | 0.44 |
| ment/control | | | | | | |
| P-value | 0.54 | 0.23 | 0.01 | 0.06 | 0.35 | 0.02 |

As is evident from the above Table 8, metformin is capable of reducing glyoxylate concentrations found in a rat model system. Accordingly, it can be assumed that metformin as a known anti-diabetic drug will also reduce the levels of the biomarker glyoxylate in diabetic patients treated by metformin. Thus, glyoxylate may presumably be used as a biomarker for monitoring the response of a diabetes patient to a metformin therapy.

1-17. (canceled)

18. A method for diagnosing diabetes or a predisposition therefor comprising:
(a) determining the amount of glyoxylate in a test sample of a subject suspected to suffer from diabetes or to have a predisposition therefor; and
(b) comparing the amount determined in step (a) to a reference, whereby diabetes or a predisposition therefor is to be diagnosed.

19. The method of claim 18, wherein said reference is derived from a subject or a group of subjects known to suffer from diabetes or to have a predisposition therefor.

20. The method of claim 19, wherein an identical or increased amount of glyoxylate in the test sample in comparison to the reference is indicative for the presence of diabetes or a predisposition therefor or wherein a decreased amount of glyoxylate in the test sample in comparison to the reference is indicative for the absence of diabetes or a predisposition therefor.

21. The method of claim 18, wherein said reference is derived from a subject or a group of subjects known to not suffer from diabetes or to not have a predisposition therefor.

22. The method of claim 21, wherein an increased amount of glyoxylate in the test sample in comparison to the reference is indicative for the presence of diabetes or a predisposition therefor or wherein an identical or decreased amount of glyoxylate in the test sample in comparison to the reference is indicative for the absence of diabetes or a predisposition therefor.

23. The method of claim 18, wherein said predisposition for diabetes is accompanied by an elevated long-term blood glucose, impaired glucose tolerance (IGT), impaired fasting glucose (IFG) or IGT in combination with IFG.

24. The method of claim 18, wherein said method further comprises the step of recommending based on the diagnosis established in step b) a therapy for the treatment or prevention of diabetes or a predisposition therefor.

25. The method of claim 18, wherein said sample is a sample of a body fluid of said subject.

26. The method of claim 18, wherein said subject is a human.

27. The method of claim 18, wherein at least one further biomarker for diabetes or a predisposition therefor is determined, wherein said at least one further biomarker is selected from the group consisting of: cryptoxanthin, 2-hydroxy-palmitic acid, triacylglyceride (C16:0, C18:1, C18:2), gondoic acid, tricosanoic acid, 5-Oxoproline, glucose, haemoglobin HbA1C, 1,5-anhydrosorbitol, 2-hydroxybutyrate, and mannose.

28. A method for diagnosing diabetes accompanied by a co-morbidity or a predisposition therefor, said method comprising:
(a) determining the amount of glyoxylate in a test sample of a subject suspected to suffer from diabetes accompanied by a co-morbidity or to have a predisposition therefor wherein said sample has been obtained from the subject during an OGTT at about 2 hours after the onset of the test; and
(b) comparing the amount determined in step (a) to a reference, whereby diabetes accompanied by a co-morbidity or a predisposition therefor is to be diagnosed.

29. A device for diagnosing diabetes or a predisposition therefor in a sample of a subject suspected to suffer therefrom comprising:
(a) an analyzing unit comprising a detection agent for glyoxylate which allows for determining the amount of glyoxylate present in the sample; and, operatively linked thereto,
(b) an evaluation unit comprising a stored reference and a data processor which allows for comparing the amount of glyoxylate determined by the analyzing unit to the stored reference, whereby diabetes or the predisposition therefor is diagnosed.

30. The device of claim 29, wherein said stored reference is a reference derived from a subject or a group of subjects known to suffer from diabetes or to have a predisposition therefor and said data processor executes instructions for comparing the amount of glyoxylate determined by the analyzing unit to the stored reference, wherein an identical or increased amount of glyoxylate in the test sample in comparison to the reference is indicative for the presence of diabetes or a predisposition therefor or wherein a decreased amount of glyoxylate in the test sample in comparison to the reference is indicative for the absence of diabetes or a predisposition therefor.

31. The device of claim 29, wherein said stored reference is a reference derived from a subject or a group of subjects known to not suffer from diabetes or to not have a predisposition therefor and said data processor executes instructions for comparing the amount of glyoxylate determined by the analyzing unit to the stored reference, wherein an increased amount of glyoxylate in the test sample in comparison to the reference is indicative for the presence of diabetes or a predisposition therefor or wherein an identical or decreased amount of glyoxylate in the test sample in comparison to the reference is indicative for the absence of diabetes or a predisposition therefor.

32. A kit for diagnosing diabetes or a predisposition therefor comprising a detection agent for glyoxylate and glyoxylate standards the concentration of which is derived from a subject or a group of subjects known to suffer from diabetes or to have a predisposition therefor or derived from a subject or a group of subjects known to not suffer from diabetes or to not have a predisposition therefor.

33. The device of claim 29, wherein said detection agent is an antibody which specifically binds to glyoxylate or an aptamere which specifically binds to glyoxylate.

34. The kit of claim 32, wherein said detection agent is an antibody which specifically binds to glyoxylate or an aptamere which specifically binds to glyoxylate.
